(19) United States
(12) Patent Application Publication
(10) Pub. No.: US 2023/0348385 A1
(43) Pub. Date: Nov. 2, 2023

(54) POLYMORPHS OF 5-BROMO-6-CHLORO-3-INDOXYL CAPRYLATE

(71) Applicant: Biosynth AG, Staad (CH)

(72) Inventors: Rith CHEAV, Thal (CH); Urs SPITZ, St. Gallen (CH); Christophe E.M. WEYMUTH, Neunkirch (CH)

(21) Appl. No.: 18/041,682

(22) PCT Filed: Aug. 17, 2021
(86) PCT No.: PCT/EP2021/072808

§ 371 Date: Feb. 14, 2023

(30) Foreign Application Priority Data
Aug. 17, 2020 (EP) 20191343.1

(51) Int. Cl. C07D 209/36

(52) U.S. Cl. CPC C07D 209/36; C07B 2200/13

(57) ABSTRACT

Polymorphs of 5-bromo-6-chloro-3-indoxyl caprylate characterized by the powder diffraction patterns as defined in FIG. 1 and FIG. 2.

### FIELD OF THE INVENTION

The present invention relates to polymorphs of 5-bromo-6-chloro-3-indoxyl caprylate, a method of making the polymorphs as well as to a system for detecting an esterase comprising a polymorph of 5-bromo-6-chloro-3-indoxyl caprylate and methods of using the polymorphs or the system.

### BACKGROUND OF THE INVENTION

5-Bromo-6-chloro-3-indoxyl caprylate (synonyms are magenta caprylate or 5-bromo-6-chloro-3-indoxyl octanoate; CAS number 209347-94-4) of formula I

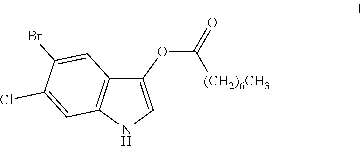

is a chromogenic substrate used, e.g., for the detection of esterase activity. The esterase enzyme cleaves the ester bond releasing 5-bromo-6-chloro-1H-indol-3-ol, which in the presence of oxygen, e.g. in air, undergoes oxidative dimerization to yield 5,5′-dibromo-6,6′-dichloro-indigo, a magenta-colored chromophore (λₘₐₓ of 565 nm). Since Salmonella, and some species of Klebsiella and Enterobacter, produce caprylate esterase, the 5-bromo-6-chloro-3-indoxyl caprylate chromogenic substrate is suitable, e.g., for the detection of pathogenic Salmonella species in food.

It is known that different crystalline solid forms, i.e. polymorphs (forms which have the same chemical composition, however, differ in the arrangement of the atoms contained therein) of active ingredients may have very different physicochemical properties. These differences may impact properties that are important for the processability and manageability thereof, such as dispersibility or solubility, dissolution rate, stability and storage. Polymorphs may also have an impact on production costs. Thus, the possible existence of polymorphs of a particular substance may be of crucial importance.

### OBJECT OF THE INVENTION

It is the object of the present invention to provide polymorphs of 5-bromo-6-chloro-3-indoxyl caprylate.

### SUMMARY OF THE INVENTION

In a first aspect, the present invention provides two polymorphs of 5-bromo-6-chloro-3-indoxyl caprylate.

In one embodiment, the invention relates to 5-bromo-6-chloro-3-indoxyl caprylate characterized by a powder diffraction pattern as defined in FIG. 1.

In another embodiment, the invention relates to 5-bromo-6-chloro-3-indoxyl caprylate characterized by a powder diffraction pattern as defined in FIG. 2.

The polymorphs may be prepared by subjecting 5-bromo-6-chloro-1H-indol-3-ol to esterification in order to obtain crude 5-bromo-6-chloro-3-indoxyl caprylate and crystallizing the crude 5-bromo-6-chloro-3-indoxyl caprylate from petroleum ether.

In a second aspect, the invention relates to a system for detecting an esterase, comprising 5-bromo-6-chloro-3-indoxyl caprylate characterized by the powder diffraction pattern as defined in FIG. 1 or FIG. 2.

In a third aspect, the invention further relates to a method of detecting the presence or absence of an esterase, comprising contacting a test sample that may comprise an esterase with 5-bromo-6-chloro-3-indoxyl caprylate characterized by the powder diffraction patterns as defined in FIG. 1 or FIG. 2, or with a system comprising 5-Bromo-6-chloro-3-indoxyl caprylate characterized by the powder diffraction pattern as defined in FIG. 1 or FIG. 2.

### BRIEF DISCUSSION OF THE FIGURES

The present invention will become more apparent by reference to the following description taken in conjunction with the following figures, in which:

FIG. 1 is a powder diffraction pattern of a polymorph of 5-bromo-6-chloro-3-indoxyl caprylate (y-axis: intensity; x-axis: position [° 2Theta] [copper (Cu)]);

FIG. 2 is a powder diffraction pattern of another polymorph of 5-bromo-6-chloro-3-indoxyl caprylate (y-axis: intensity; x-axis: position [° 2Theta] [copper (Cu)]);

FIG. 3 is a comparison of the powder diffraction patterns of FIG. 1 and FIG. 2;

FIG. 4 is a DSC scans of the first and the second polymorph in comparison (y-axis: power; x-axis: temperature and scan rate of 10 K/min); a is the first run of the polymorph characterized by the powder diffraction pattern of FIG. 1 and b the second run thereof; c is the first run of the polymorph characterized by the powder diffraction pattern of FIG. 2 and d the second run thereof;

FIG. 5 is a SEM (Scanning Electron Microscope) recording of 5-bromo-6-chloro-3-indoxyl caprylate of FIG. 1 at a magnification of 497:1;

FIG. 6 is a SEM recording of 5-bromo-6-chloro-3-indoxyl caprylate of FIG. 2 at a magnification of 199:1.

### DETAILED DESCRIPTION

In the following detailed description of the present invention, specific details are set forth in order to provide a more thorough understanding of the invention. In other instances, well-known features have not been described in detail to avoid obscuring the invention.

### First Aspect of the Invention

Although not limited thereto, the compound of formula I may be e.g. prepared by esterification of 5-bromo-6-chloro-1H-indol-3-ol with caprylic acid chloride according to known methods.

In one embodiment, the compound of formula I obtained in the esterification of 5-bromo-6-chloro-1H-indol-3-ol is an amorphous powder.

In another embodiment, the compound of formula I obtained in the esterification of 5-bromo-6-chloro-1H-indol-3-ol is semi-crystalline.

In still another embodiment, the compound of formula I obtained in the esterification of 5-bromo-6-chloro-1H-indol-3-ol is a mixture of amorphous powder and crystals.

Desirably, the compound of formula I obtained after esterification, i.e. crude 5-bromo-6-chloro-3-indoxyl caprylate, has to be purified in order to be suitable for the targeted application. A preferred purification method is crystallization.

The term “crystallization” as used in this disclosure is synonymously used with the term “re-crystallization”.

It has been unexpectedly discovered that 5-bromo-6-chloro-3-indoxyl caprylate, when subjected to a crystallization step according to the present invention, may be isolated in the form of at least two polymorphs.

Preferably, the solvent used for the crystallization is an aprotic and non-polar solvent.

The term “aprotic” is used in this disclosure in its common meaning, i.e. the solvent is not capable to dissociate upon forming protons.

The term “non-polar” is used in this disclosure in its common meaning, i.e. the molecules of the solvent do not possess a permanent dipole moment.

Preferably, the aprotic and non-polar solvent comprises or consists of one or more hydrocarbons.

Suitable hydrocarbons are pentanes, hexanes, heptanes, octanes, nonanes and still higher alkanes and mixtures of two or more thereof.

Preferably, hydrocarbons are pentanes, hexanes, heptanes, octanes, and nonanes and mixtures of two or more thereof.

In a further preferred embodiment, the solvent is selected from a petroleum ether.

The term “petroleum ether” as used in this disclosure denotes a mixture of different alkanes such as pentanes, hexanes and heptanes, preferably a mixture of pentanes and hexanes.

In one embodiment, the aprotic and non-polar solvent, preferably the petroleum ether, has a boiling point in the range of from 40 to 100° C.

In another embodiment, the aprotic and non-polar solvent, preferably the petroleum ether, has a boiling point in the range of from 25 to 80° C.

After crystallization in the solvent, crystalline 5-bromo-6-chloro-3-indoxyl caprylate may be isolated according to known methods, e.g. by filtration and subsequent drying.

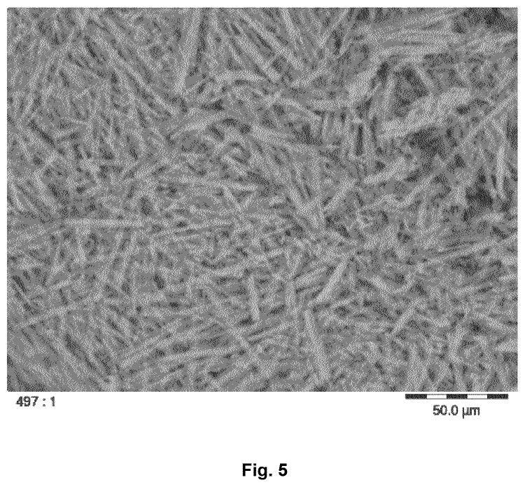

In one embodiment, 5-bromo-6-chloro-3-indoxyl caprylate obtained in the crystallization method according to the present invention is in the form of a microcrystalline and compact form. FIG. 5 shows a SEM (Scanning Electron microscope) recording of this polymorph.

This polymorph has a melting point of 63° C.±1° C. determined using a differential scanning calorimeter (DSC) at a scan rate of 10.0 k/min according to ASTM D 3418.

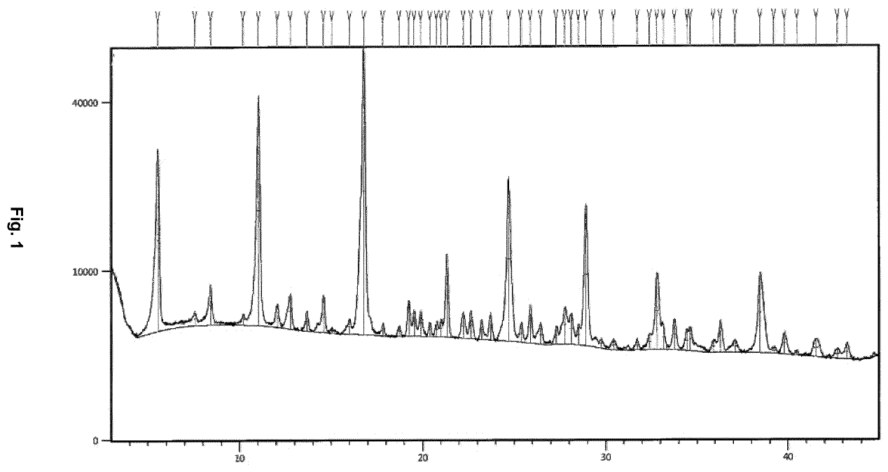

This polymorph of 5-bromo-6-chloro-3-indoxyl caprylate is characterized by a powder diffraction pattern as defined in FIG. 1.

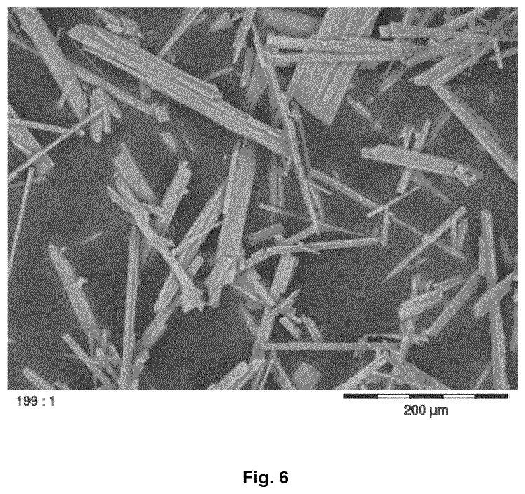

In another preferred embodiment, 5-bromo-6-chloro-3-indoxyl caprylate is obtained after crystallization in the form of a cotton-like and voluminous form. FIG. 6 shows a SEM recording of this polymorph.

This polymorph has a melting point of 60° C.±1° C. determined using a differential scanning calorimeter (DSC) at a scan rate of 10.0 k/min according to ASTM D 3418.

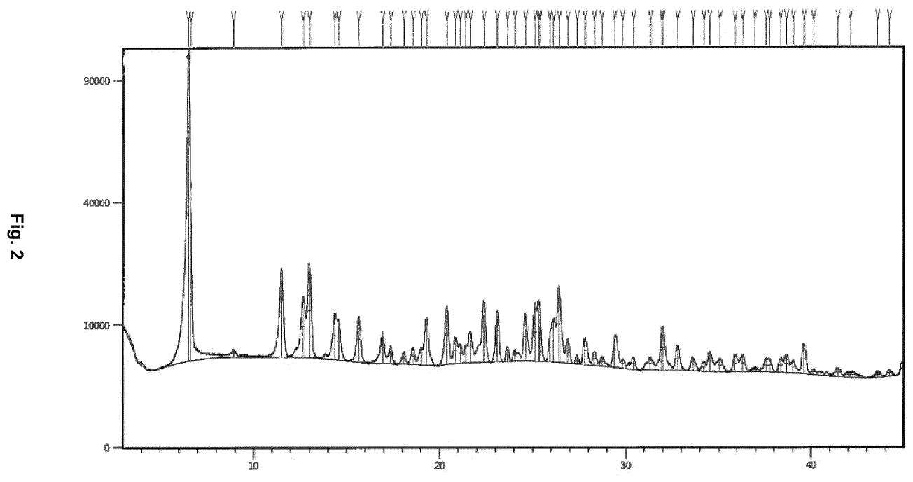

This polymorph of 5-bromo-6-chloro-3-indoxyl caprylate is characterized by a powder diffraction pattern as defined in FIG. 2.

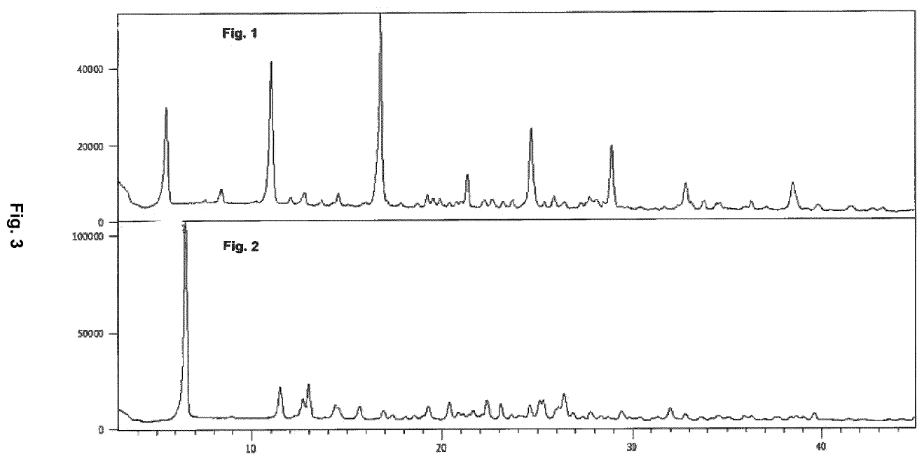

FIG. 3 shows a comparison of the respective powder diffraction patterns.

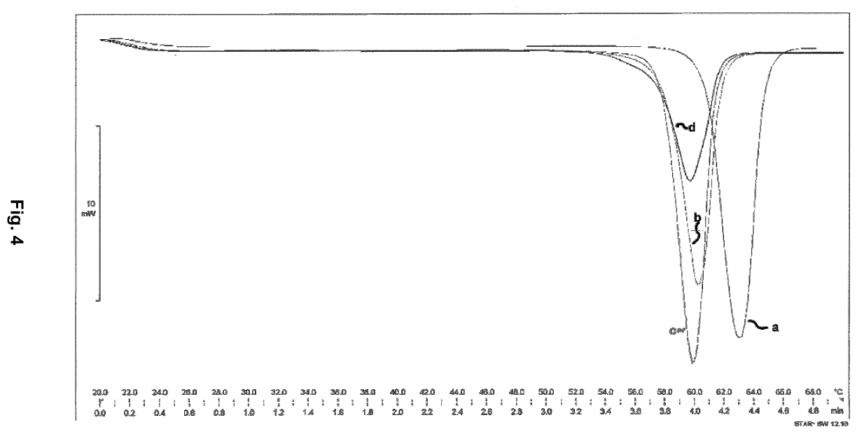

Without being bound by theory, it is believed that the polymorph characterized by the powder diffraction diagram of FIG. 2 is the thermodynamically stable form since the polymorph characterized by the powder diffraction pattern defined in FIG. 1 can be converted into the polymorph characterized by the powder diffraction pattern of FIG. 2 by melting. FIG. 4 shows this when the polymorph characterized by the powder diffraction pattern according to FIG. 1 is heated in a DSC in order to determine the melting point (curve a), and subsequently is cooled down and re-melted and solidified (curve b). Accordingly, the polymorph characterized by the powder diffraction pattern of FIG. 1 probably represents a metastable polymorph.

In one embodiment, 5-bromo-6-chloro-3-indoxyl caprylate characterized by a powder diffraction pattern as defined in FIG. 1 can be obtained by cooling down a petroleum ether solution saturated with the compound of formula I to a temperature of from 24° C. to 26° C. and isolating the formed crystals. Preferably, this cooling down is performed slowly.

In another embodiment, 5-bromo-6-chloro-3-indoxyl caprylate characterized by a powder diffraction pattern as defined in FIG. 2 can be obtained by cooling down a saturated petrol ether solution to a temperature below 24° C. to 26° C. and isolating the formed crystals. Preferably, this cooling down is performed fast.

Accordingly, the first aspect also encompasses a method of making 5-bromo-6-chloro-3-indoxyl caprylate characterized by a powder diffraction method defined in FIG. 1 or FIG. 2, comprising: Subjecting 5-bromo-6-chloro-1H-indol-3-ol to esterification in order to obtain crude 5-bromo-6-chloro-3-indoxyl caprylate and crystallizing the crude 5-bromo-6-chloro-3-indoxyl caprylate from petroleum ether.

Characteristic peaks of the polymorph characterized by the powder diffraction pattern defined in FIG. 1 are recorded in Table 1:

TABLE 1

| Pos. | Height | FWHM Left | d-spacing | Rel. Int. |
|---|---|---|---|---|
| [°2Th.] | [cts] | [°2Th.] | [Å] | [%] |
| 5.5376 | 25513.71 | 0.1151 | 15.95935 | 50.55 |
| 7.5584 | 1169.66 | 0.1023 | 11.69648 | 2.32 |
| 8.4187 | 3754.91 | 0.1279 | 10.50313 | 7.44 |
| 10.2075 | 942.16 | 0.1023 | 8.66613 | 1.87 |
| 11.0389 | 37076.81 | 0.1407 | 8.01525 | 73.46 |
| 12.0703 | 2093.44 | 0.0640 | 7.33259 | 4.15 |
| 12.7692 | 3265.13 | 0.1279 | 6.93279 | 6.47 |
| 13.6869 | 1630.47 | 0.1151 | 6.46991 | 3.23 |
| 14.5860 | 3266.43 | 0.1151 | 6.07309 | 6.47 |
| 15.0550 | 369.01 | 0.2047 | 5.88492 | 0.73 |
| 16.0015 | 1256.40 | 0.1023 | 5.53890 | 2.49 |
| 16.7875 | 50473.18 | 0.1407 | 5.28129 | 100.00 |
| 17.8347 | 957.92 | 0.1023 | 4.97348 | 1.90 |
| 18.7199 | 792.65 | 0.1535 | 4.74025 | 1.57 |
| 19.2462 | 3044.08 | 0.1023 | 4.61181 | 6.03 |
| 19.5344 | 2105.44 | 0.0640 | 4.54441 | 4.17 |
| 19.8926 | 2160.67 | 0.0895 | 4.46338 | 4.28 |
| 20.3976 | 1099.47 | 0.1151 | 4.35400 | 2.18 |
| 20.7706 | 1214.26 | 0.1279 | 4.27665 | 2.41 |
| 21.0192 | 1346.51 | 0.1023 | 4.22661 | 2.67 |
| 21.3302 | 8489.90 | 0.1279 | 4.16570 | 16.82 |
| 22.2201 | 1949.07 | 0.1279 | 4.00084 | 3.86 |
| 22.6480 | 2250.79 | 0.0640 | 3.92621 | 4.46 |
| 23.2283 | 1476.95 | 0.0895 | 3.82942 | 2.93 |
| 23.7132 | 2091.51 | 0.1023 | 3.75219 | 4.14 |
| 24.7074 | 20723.83 | 0.1279 | 3.60342 | 41.06 |
| 25.3948 | 1542.02 | 0.1279 | 3.50742 | 3.06 |
| 25.8847 | 3151.10 | 0.1023 | 3.44214 | 6.24 |
| 26.4467 | 1433.38 | 0.1535 | 3.37025 | 2.84 |
| 27.3052 | 1319.69 | 0.1023 | 3.26620 | 2.61 |
| 27.7696 | 3047.14 | 0.0768 | 3.21263 | 6.04 |
| 28.1095 | 2379.87 | 0.1535 | 3.17455 | 4.72 |
| 28.5271 | 1520.60 | 0.1023 | 3.12903 | 3.01 |
| 28.9194 | 16338.76 | 0.1663 | 3.08747 | 32.37 |
| 29.7498 | 612.81 | 0.1535 | 3.00315 | 1.21 |
| 30.4293 | 629.40 | 0.2558 | 2.93762 | 1.25 |
| 31.7195 | 661.72 | 0.1023 | 2.82102 | 1.31 |
| 32.4181 | 1070.99 | 0.1279 | 2.76180 | 2.12 |
| 32.8161 | 6893.78 | 0.1535 | 2.72921 | 13.66 |
| 33.1576 | 1789.13 | 0.1023 | 2.70188 | 3.54 |
| 33.7673 | 2229.99 | 0.1151 | 2.65447 | 4.42 |
| 34.4493 | 1485.39 | 0.1279 | 2.60347 | 2.94 |
| 34.6259 | 1676.34 | 0.1023 | 2.59060 | 3.32 |
| 35.9047 | 800.50 | 0.1535 | 2.50121 | 1.59 |
| 36.2823 | 2385.33 | 0.1279 | 2.47604 | 4.73 |
| 37.0742 | 788.63 | 0.1791 | 2.42495 | 1.56 |
| 38.4596 | 7133.31 | 0.1151 | 2.34073 | 14.13 |
| 39.2177 | 393.67 | 0.1535 | 2.29721 | 0.78 |
| 39.7913 | 1541.51 | 0.1279 | 2.26541 | 3.05 |
| 40.4590 | 266.97 | 0.1535 | 2.22955 | 0.53 |

Characteristic peaks of the polymorph characterized by the powder diffraction pattern defined in FIG. 2 are recorded in Table 2:

TABLE 2

| Pos. | Height | FWHM Left | d-spacing | Rel. Int. |
|---|---|---|---|---|
| [°2Th.] | [cts] | [°2Th.] | [Å] | [%] |
| 6.5175 | 102212.90 | 0.1023 | 13.56213 | 100.00 |
| 6.6308 | 39277.21 | 0.0512 | 13.33060 | 38.43 |
| 8.9286 | 961.50 | 0.1535 | 9.90439 | 0.94 |
| 11.5021 | 16055.72 | 0.1407 | 7.69349 | 15.71 |
| 12.6789 | 9837.40 | 0.1407 | 6.98193 | 9.62 |
| 12.9985 | 17456.36 | 0.1279 | 6.81096 | 17.08 |
| 14.3767 | 6993.04 | 0.1023 | 6.16100 | 6.84 |
| 14.6039 | 5406.42 | 0.1535 | 6.06566 | 5.29 |
| 15.6513 | 6558.35 | 0.1279 | 5.66204 | 6.42 |
| 16.9316 | 4373.57 | 0.1279 | 5.23667 | 4.28 |
| 17.3772 | 2092.36 | 0.1151 | 5.10337 | 2.05 |
| 18.0760 | 1412.13 | 0.1279 | 4.90764 | 1.38 |
| 18.5533 | 2070.24 | 0.1023 | 4.78244 | 2.03 |
| 19.0140 | 2054.71 | 0.1535 | 4.66760 | 2.01 |
| 19.2918 | 6812.08 | 0.1279 | 4.60099 | 6.66 |
| 20.3781 | 8838.61 | 0.1023 | 4.35812 | 8.65 |
| 20.8525 | 3360.93 | 0.1151 | 4.26003 | 3.29 |
| 21.0979 | 2391.37 | 0.1023 | 4.21103 | 2.34 |
| 21.3976 | 2304.30 | 0.1023 | 4.15273 | 2.25 |
| 21.6361 | 4231.65 | 0.1407 | 4.10749 | 4.14 |
| 22.3646 | 9474.15 | 0.1663 | 3.97532 | 9.27 |
| 23.0879 | 7568.22 | 0.1791 | 3.85238 | 7.40 |
| 23.6352 | 1811.11 | 0.1279 | 3.76440 | 1.77 |
| 24.0357 | 1351.87 | 0.1279 | 3.70257 | 1.32 |
| 24.6134 | 6944.84 | 0.1407 | 3.61696 | 6.79 |
| 25.1249 | 9121.06 | 0.1535 | 3.54448 | 8.92 |
| 25.3109 | 9316.60 | 0.0780 | 3.51594 | 9.11 |
| 25.3814 | 8236.42 | 0.0640 | 3.50924 | 8.06 |
| 25.9218 | 3656.63 | 0.0768 | 3.43730 | 3.58 |
| 26.1124 | 6242.41 | 0.0640 | 3.41263 | 6.11 |
| 26.4193 | 12612.88 | 0.1919 | 3.37368 | 12.34 |
| 26.8802 | 3176.88 | 0.1663 | 3.31687 | 3.11 |
| 27.3671 | 961.55 | 0.1535 | 3.25896 | 0.94 |
| 27.8189 | 3565.15 | 0.1535 | 3.20705 | 3.49 |
| 28.3266 | 1594.11 | 0.1535 | 3.15071 | 1.56 |
| 28.7231 | 1066.77 | 0.1535 | 3.10812 | 1.04 |
| 29.4019 | 3810.05 | 0.0768 | 3.03789 | 3.73 |
| 29.8329 | 1048.47 | 0.1535 | 2.99498 | 1.03 |
| 30.4091 | 1351.44 | 0.1279 | 2.93952 | 1.32 |
| 31.3182 | 1482.25 | 0.1535 | 2.85624 | 1.45 |
| 31.9404 | 5329.56 | 0.0936 | 2.79969 | 5.21 |
| 32.0017 | 5909.08 | 0.0768 | 2.79678 | 5.78 |
| 32.7997 | 2862.93 | 0.1791 | 2.73053 | 2.80 |
| 33.6102 | 1511.81 | 0.2303 | 2.66652 | 1.48 |
| 34.2052 | 974.97 | 0.1279 | 2.62149 | 0.95 |
| 34.5399 | 2345.48 | 0.1791 | 2.59685 | 2.29 |
| 35.0704 | 1472.83 | 0.2047 | 2.55877 | 1.44 |
| 35.8845 | 2007.99 | 0.0895 | 2.50257 | 1.96 |
| 36.2983 | 1993.39 | 0.1279 | 2.47498 | 1.95 |
| 36.9614 | 420.85 | 0.2558 | 2.43209 | 0.41 |

### Second Aspect of the Invention:

According to a second aspect, the present invention relates to a system for detecting an esterase.

The systems that use chromogenic compounds such as the compound of formula I are typically plates for bacteriology filled with an aqueous culture media composed by classic ingredients like agar and various organic or salt nutrients suitable to the growth of the microorganisms comprising said esterase. Such systems are known in the art.

Accordingly, the present invention further relates to a system for detecting an esterase, comprising a polymorph of 5-bromo-6-chloro-3-indoxyl caprylate as defined in the first aspect.

In a preferred embodiment, said esterase is produced by Salmonella species.

### Third Aspect of the Invention

According to a third aspect, the present invention relates to a method of detecting the presence or absence of an esterase. The basic principles of such method are as discussed above in the BACKGROUND section.

Accordingly, the present invention relates to a method of detecting the presence or absence of an esterase, comprising contacting a test sample that may comprise an esterase with a polymorph as defined in the first aspect or with a system as defined in the third aspect.

The test sample may be a bacterium such as Salmonella species or a substrate comprising a bacterium.

1. 5-Bromo-6-chloro-3-indoxyl caprylate characterized by a powder diffraction pattern as defined in FIG. 1.

2. 5-Bromo-6-chloro-3-indoxyl caprylate according to claim 1 having a melting point of 63° C.±1° C. determined by DSC at a scan rate of 10.0 K/m in according to ASTM D 3418.

3. 5-Bromo-6-chloro-3-indoxyl caprylate characterized by a powder diffraction pattern as defined in FIG. 2.

4. 5-Bromo-6-chloro-3-indoxyl caprylate according to claim 3 having a melting point of 60° C.±1° C. determined by DSC at a scan rate of 10.0 K/m in according to ASTM D 3418.

5. Method of making 5-bromo-6-chloro-3-indoxyl caprylate according to claim 1 or 3, comprising: Subjecting 5-bromo-6-chloro-1H-indol-3-ol to esterification in order to obtain crude 5-bromo-6-chloro-3-indoxyl caprylate and crystallizing the crude 5-bromo-6-chloro-3-indoxyl caprylate from petroleum ether.

6. A system for detecting an esterase, comprising 5-bromo-6-chloro-3-indoxyl caprylate as defined in claim 1 or 3, or 5-bromo-6-chloro-3-indoxyl caprylate obtained in the method according to claim 5.

7. A method of detecting the presence or absence of an esterase, comprising contacting a test sample that may comprise an esterase with a compound defined in claim 1 or 3 or with a system as defined in claim 5.
